(19) United States
(12) Patent Application Publication
(10) Pub. No.: US 2004/0030280 A1
(43) Pub. Date: Feb. 12, 2004

(54) Leak resistant tampon system

(76) Inventor: Thomas G. Mercier, Houghton, MI (US)

Correspondence Address: Michael S. Neustel, Suite No. 4, 2534 South University Drive, Fargo, ND, 58103, US

(21) Appl. No.: 10/217,268

(22) Filed: Aug. 8, 2002

(51) Int. Cl. A61F 13/20; A61F 13/15

(52) U.S. Cl. 604/11; 604/904

(57) ABSTRACT

A leak resistant tampon system for increasing absorbency capacity and reducing leakage about a tampon device. The leak resistant tampon system includes a housing member having a front end and a front opening, a front collecting reservoir fluidly connected to the front opening, and a volume of highly absorbent material throughout but particularly within the housing member fluidly connected to the front collecting reservoir. The absorbent material preferably has a porous fluid permeable central channel within for increasing the overall absorption capabilities and capacity of the absorbent material. A plurality of annular ribs and concave grooves may extend from the outer surface of the housing member for creating a seal increasing absorbency surface area and efficiency when positioned within the vagina.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to tampon devices and more specifically it relates to a leak resistant tampon system for reducing leakage about a tampon device.

2. Description of the Related Art

Tampon devices have been in use for years. Conventional tampons are comprised of a plug of absorbent material that is inserted into the vagina to check and absorb blood and secretions during menstruation. Tampons are often times comprised of a rigid tubular structure with an absorbent material within such as cotton that absorbs bodily fluids.

The main problem with conventional tampon devices is that they are prone to leakage because of lack of sealing and lack of proper absorption. A further problem with conventional tampon devices is that they typically utilize a solid or semi-porous tip which does not absorb fluid fast enough thereby forcing the bodily fluid to pass about the outside of the tampon. A further problem is once the frontal tip of a conventional tampon becomes saturated it is unable to absorb additional fluids, thereby causing fluid to pass about the outside of the tampon.

Examples of patented devices which may be related to the present invention include U.S. Pat. No. 6,177,608 to Weinstrauch; U.S. Pat. No. 5,772,645 to Zadini et al.; U.S. Pat. No. 5,718,675 to Leijd; U.S. Pat. No. 4,274,412 to Austin; U.S. Pat. No. 4,341,215 to Eldridge; U.S. Pat. No. 4,335,720 to Glassman; U.S. Pat. No. 4,175,561 to Hirschman; U.S. Pat. No. 4,027,673 to Poncy et al.; U.S. Pat. No. 4,020,841 to Poncy et al.; U.S. Pat. No. 3,946,737 to Kobler; U.S. Pat. No. 3,683,915 to Voss.

While these devices may be suitable for the particular purpose to which they address, they are not as suitable for reducing leakage about a tampon device. Conventional tampon devices are prone to leakage because of lack of sealing and lack of absorption capabilities.

In these respects, the leak resistant tampon system according to the present invention substantially departs from the conventional concepts and designs of the prior art, and in so doing provides an apparatus primarily developed for the purpose of reducing leakage about a tampon device.

### BRIEF SUMMARY OF THE INVENTION

In view of the foregoing disadvantages inherent in the known types of tampon devices now present in the prior art, the present invention provides a new leak resistant tampon system construction wherein the same can be utilized for increasing absorbency and reducing leakage about a tampon device.

The general purpose of the present invention, which will be described subsequently in greater detail, is to provide a new leak resistant tampon system that has many of the advantages of the tampon devices mentioned heretofore and many novel features that result in a new leak resistant tampon system which is not anticipated, rendered obvious, suggested, or even implied by any of the prior art tampon devices, either alone or in any combination thereof.

To attain this, the present invention generally comprises a housing member having a front end and a front opening, a front collecting reservoir fluidly connected to the front opening, and a volume of highly absorbent material throughout but particularly within the housing member fluidly connected to the front collecting reservoir. The absorbent material preferably has a porous and fluid permeable central channel within for increasing the overall absorption capabilities of the absorbent material. A plurality of annular ribs and concave grooves may extend from the outer surface of the housing member for creating a seal, increasing absorbency surface area and efficiency when positioned within the vagina.

There has thus been outlined, rather broadly, the more important features of the invention in order that the detailed description thereof may be better understood, and in order that the present contribution to the art may be better appreciated. There are additional features of the invention that will be described hereinafter and that will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a leak resistant tampon system that will overcome the shortcomings of the prior art devices.

A second object is to provide a leak resistant tampon system for reducing leakage about a tampon device.

Another object is to provide a leak resistant tampon system that has an increased absorption capacity rate.

An additional object is to provide a leak resistant tampon system that increases the effectively absorption surface area.

A further object is to provide a leak resistant tampon system that improves sealing within a vagina.

Another object is to provide a leak resistant tampon system that has a front collection and reservoir structure.

A further object is to provide a leak resistant tampon system that is comprised of a flexible structure.

Other objects and advantages of the present invention will become obvious to the reader and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention.

FIG. 2 is a side view of the present invention.

FIG. 3 is a side view of the present invention positioned within the vagina.

FIG. 4 is a cross sectional view taken along line 4-4 of FIG. 2.

FIG. 5 is a front end view of the present invention.

### DETAILED DESCRIPTION OF THE INVENTION

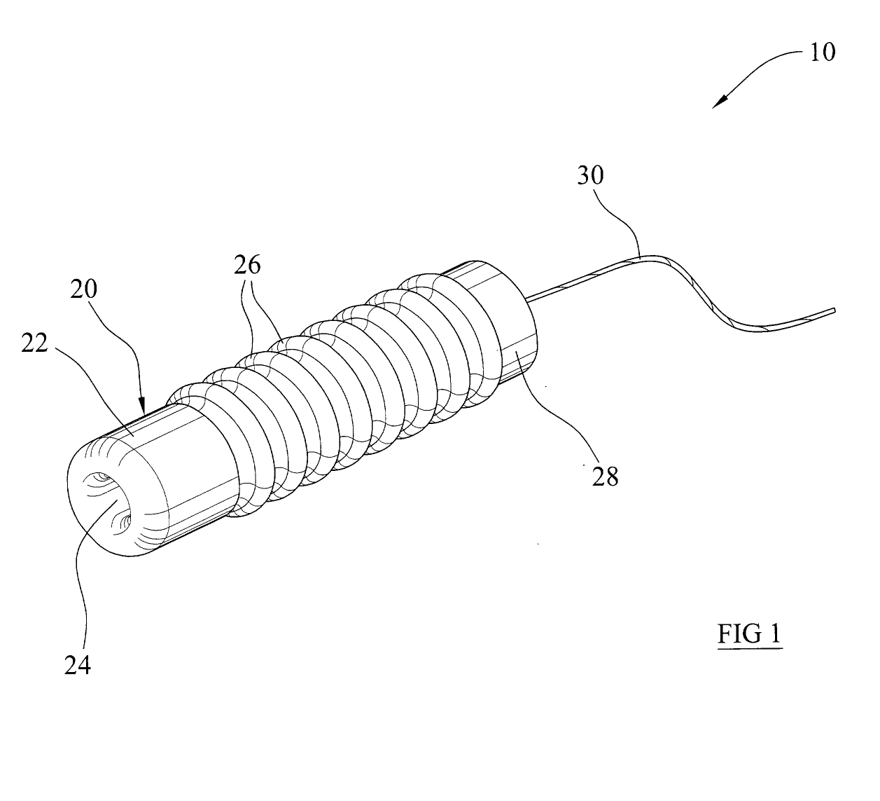

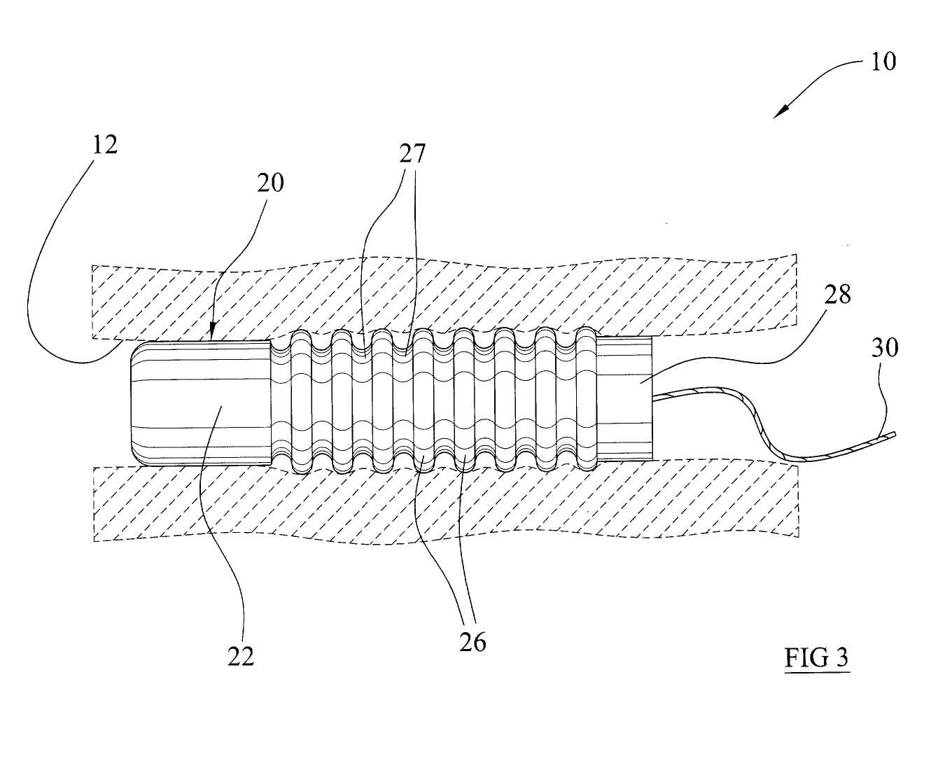

Turning now descriptively to the drawings, in which similar reference characters denote similar elements throughout the several views, FIGS. 1 through 6 illustrate a leak resistant tampon system 10, which comprises a housing member 20 having a front end 22 and a front opening 24, a front collecting reservoir 25 fluidly connected to the front opening 24, and a volume of highly absorbent material 40 within the housing member 20 fluidly connected to the front collecting reservoir 25. The absorbent material 40 preferably has a porous and fluid permeable central channel 42 within for increasing the overall absorption capabilities of the highly absorbent material 40. A plurality of annular ribs 26 may extend from the outer surface of the housing member 20 for creating a seal and increasing total absorbing surface area when positioned within the vagina 12. A plurality of circular concave grooves 27 between the plurality of annular ribs 26 help trap fluids, increase total absorption surface area and aid in sealing. The present invention is preferably entirely comprised of an absorbing material, including the housing member 20, however various other structure includes partially non-absorbent material may also be utilized.

As shown in FIGS. 1 through 5 of the drawings, the housing member 20 is preferably an elongate structure. The housing member 20 preferably has a circular cross sectional shape, however various other cross sectional shapes may be utilized to construct the housing member 20. The housing member 20 is preferably comprised of a fluid absorbing material for absorbing bodily fluids within the vagina 12, however the housing member 20 may be comprised of a non-absorbing material or semi-absorbing material. The housing may also have an outer coating.

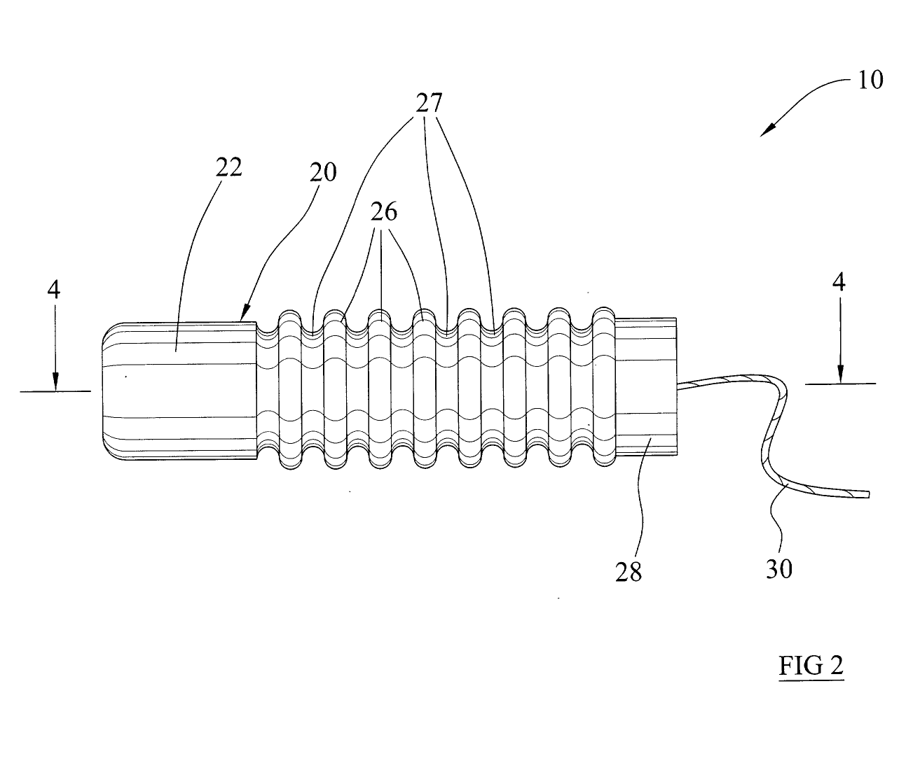

The housing member 20 has a front end 22 and a rear end 28 as best illustrated in FIG. 2 of the drawings. The exterior surface of the housing member 20 may have a consistent and straight structure or a plurality of annular ribs 26 may extend from the housing member 20 for sealing within the vagina 12. A plurality of concave grooves 27 are preferably formed between the annular ribs 26 as best illustrated in FIGS. 2 through 4 of the drawings.

The plurality of annular ribs 26 and concave grooves 27 may extend through the entire length of the housing member 20 or only a portion thereof such as centrally. If the annular ribs 26 and concave grooves 27 only cover a portion of the exterior surface of the housing member 20, the front end 22 and the rear end 28 preferably have a relatively flat exterior surface as best illustrated in FIGS. 1 through 4 of the drawings.

As shown in FIGS. 1 through 4 of the drawings, a cord member 30 having a specific length is attached to the rear end 28 of the housing member 20 for assisting in removal of the housing member 20 from the vagina 12. The cord member 30 may have various lengths and may be constructed from various types of materials commonly utilized to construct cords. The cord member 30 may also be attached to the housing member 20 utilizing various securing systems such as but not limited to molded within the housing member 20, fasteners, or adhesive.

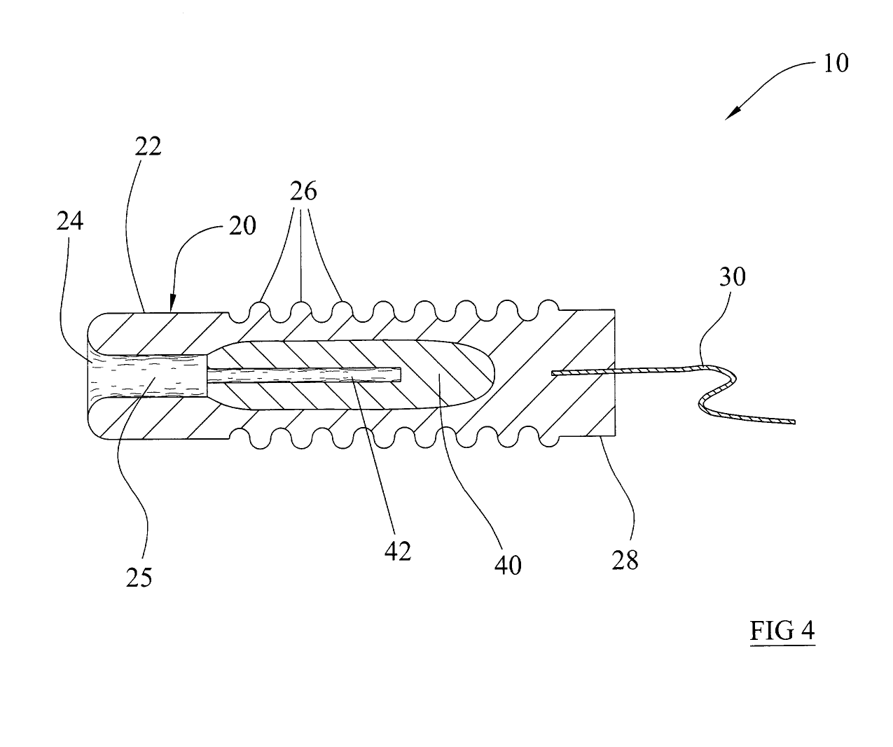

As shown in FIGS. 1, 4 and 5 of the drawings, the front end 22 of the housing member 20 has a front opening 24 within for receiving fluids within the vagina 12. The front opening 24 may have various sizes and shapes. However, it is desirable to have the initial portion of the front opening 24 relatively larger and then angled/curved inwardly for receiving fluids along the inner walls of the vagina 12 as best illustrated in FIG. 4 of the drawings.

A front collecting reservoir 25 is positioned within the housing member 20 and is fluidly connected to the front opening 24. The front collecting reservoir 25 is formed for receiving and absorbing a volume of bodily fluid not absorbed by the absorbent material 40 thereby reducing the likelihood of passing about the exterior of the housing member 20. The front collecting reservoir 25 preferably has a smaller cross section than the front opening 24, however various shapes and sizes may be utilized to construct the front collecting reservoir 25.

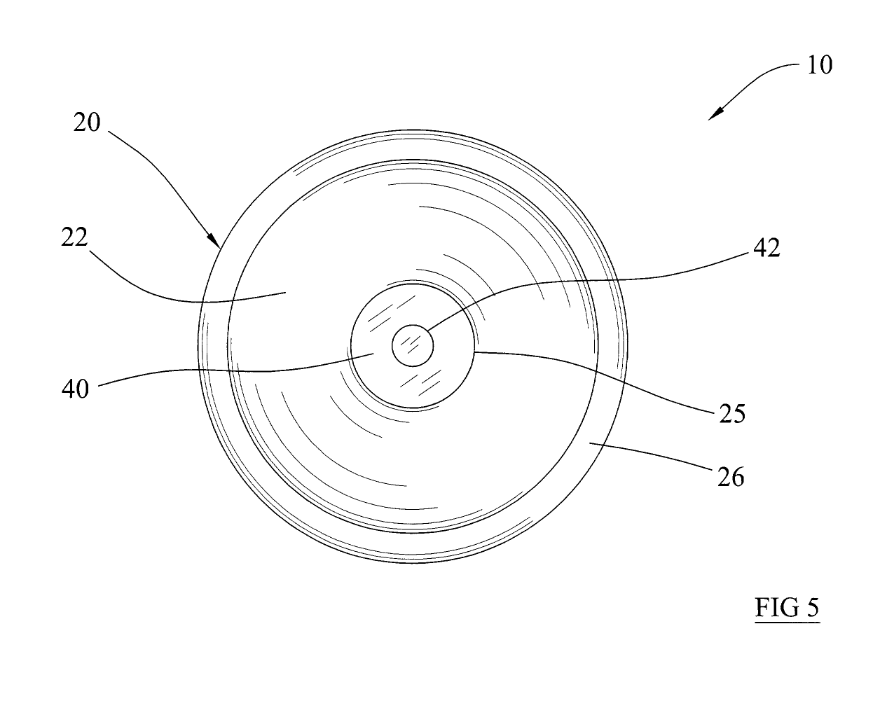

As shown in FIGS. 4 and 5 of the drawings, the entire tampon is made up of absorbing materials and a volume of absorbent material 40 is positioned within the housing member 20 in a fluidly connected manner to the front collecting reservoir 25. The absorbent material 40 is preferably comprised of a high fluid absorbing material capable of receiving and storing a relatively significant amount of bodily fluid commonly utilized within the diaper industry such as but not limited to cellulose or cotton. Additional fluid absorbing materials will be attached to the absorbent material 40 such as but not limited to sodium polyacrylate. As shown in FIG. 4 of the drawings, the highly absorbent material 40 preferably extends along a significant length of the housing member 20 and preferably 50% of the length of the housing member 20.

As further shown in FIG. 4 of the drawings, a porous and fluid permeable hollowed out channel 42 preferably extends through the absorbent material 40 from the front collecting reservoir 25. The channel 42 may extend either partially or completely through the absorbent material 40. The channel 42 may have various shapes and sizes capable of allowing bodily fluid to flow and be absorbed throughout the entire 40 thereby increasing the overall absorbing surface area of the absorbent material 40 thereby increasing the absorbent capabilities and capacity along and throughout the absorbent material 40. The channel 42 is preferably centrally positioned within the absorbent material 40 thereby increasing the volume of absorbent material 40 surrounding the channel 42.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed to be within the expertise of those skilled in the art, and all equivalent structural variations and relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A tampon system, comprising:
a housing member having a front end;
a front opening within said front end of said housing member;
an absorbent material within said housing member fluidly connected to said front opening, wherein said absorbent material is capable of absorbing and retaining a volume of fluid; and
a channel extending into said absorbent material from said front opening.

2. The tampon system of claim 1, wherein said channel extends centrally into said absorbent material.

3. The tampon system of claim 1, wherein said channel extends partially into said absorbent material.

4. The tampon system of claim 1, wherein said channel extends completely through said absorbent material.

5. The tampon system of claim 1, wherein said absorbent material extends along at least 50% of a length of said housing member.

6. The tampon system of claim 1, wherein said housing member is comprised of a fluid absorbing material.

7. The tampon system of claim 1, including a plurality of annular ribs extending from an exterior surface of said housing member defining a plurality of concave grooves between thereof.

8. The tampon system of claim 7, wherein said annular ribs extend along a middle portion of said housing member.

9. The tampon system of claim 1, wherein said front opening is comprised of a tapered structure.

10. The tampon system of claim 1, including a cord member attached to a rear end of said housing member.

11. A tampon system, comprising:
a housing member having a front end;
a front opening within said front end of said housing member;
a front collecting reservoir within said housing member fluidly connected to said front opening capable of receiving a volume of fluid;
an absorbent material within said housing member fluidly connected to said front collecting reservoir opposite of said front opening, wherein said absorbent material is capable of absorbing and retaining a volume of fluid; and
a channel extending into said absorbent material from said front opening.

12. The tampon system of claim 11, wherein said channel extends centrally into said absorbent material.

13. The tampon system of claim 11, wherein said channel extends partially into said absorbent material.

14. The tampon system of claim 11, wherein said channel extends completely through said absorbent material.

15. The tampon system of claim 11, wherein said absorbent material extends along at least 50% of a length of said housing member.

16. The tampon system of claim 11, wherein said housing member is comprised of a fluid absorbing material.

17. The tampon system of claim 11, including a plurality of annular ribs extending from an exterior surface of said housing member defining a plurality of concave grooves between thereof.

18. The tampon system of claim 17, wherein said annular ribs extend along a middle portion of said housing member.

19. The tampon system of claim 11, wherein said front opening is comprised of a tapered structure.

20. The tampon system of claim 11, including a cord member attached to a rear end of said housing member.
